(19) United States
(12) Patent Application Publication
(10) Pub. No.: US 2011/0256089 A1
(43) Pub. Date: Oct. 20, 2011

(54) Hydrogel Type Cell Delivery Vehicle for Wound Healing, and Preparation Method Thereof

(75) Inventors: Sae-Hwan Lim, Seoul (KR); Yun Young Kim, Gyeonggi-do (KR); So Hee Yun, Seoul (KR)

(73) Assignee: MODERN CELL & TISSUE TECHNOLOGIES INC., Nowon-gu, Seoul (KR)

(21) Appl. No.: 13/127,165

(22) PCT Filed: Nov. 3, 2009
(86) PCT No.: PCT/KR09/06425

§ 371 Date: Jun. 8, 2011

(30) Foreign Application Priority Data
Nov. 3, 2008 (KR) 1020080108458

(51) Int. Cl. A61K 38/19; A61K 38/18; A61K 38/30; A61K 38/39; A61K 35/12; A61K 31/726; A61K 35/36; A61K 35/30; A61K 35/28; A61K 47/34; A61K 31/728

(52) U.S. Cl. 424/85.1; 514/788; 514/784; 514/9.6; 514/9.1; 514/8.5; 514/7.6; 514/17.2; 514/54; 514/9.3; 424/93.7

(57) ABSTRACT

Disclosed is a hydrogel type cell delivery vehicle composition for wound healing and to a preparation method thereof. More particularly, the present invention relates to a hydrogel type cell delivery vehicle composition in which non-ionic surfactants, growth factors or substance-P, human-derived cells, and the like are distributed in aqueous media, to a use thereof for wound healing, and to a preparation method thereof. The hydrogel type composition of the present invention appropriately delivers cells and/or substance-P to the wound part, and has moistening effects, effects of preventing contraction of the wound, and effects of protecting cells, and can be used in an easy and convenient manner. The cells in the composition are delivered to the wound part to effectively heal the wound when the composition of the present invention is applied to or injected into the wounded body part.

### BACKGROUND OF THE INVENTION

1. Field of the Invention

The present invention relates to a hydrogel-type cell delivery vehicle composition for wound healing and a method for the preparation thereof. More particularly, the present invention relates to a hydrogel-type cell delivery vehicle composition comprising an aqueous medium in which a non-ionic surfactant is dispersed alone or in combination with a growth factor Substance-P or cells, to the use of the vehicle composition in wound healing, and a method for the preparation thereof.

2. Description of the Related Art

Tissue reconstruction for wounds has been extensively studied for a long time. Tissue reconstruction is typically conducted with drugs and/or cells. However, important points in relation to the delivery of these drugs and cells to injured tissues are how the drugs are delivered and what their composition is. For use in delivery to a tissue of interest, drugs and cells may be formulated simply into a solution, or further formed as a sheet, a sponge or a non-woven fabric in combination with a biomaterial such as collagen, or combined with a fibrin adhesive.

Substance-P is a neuropeptide consisting of 11 amino acid residues and is known to be expressed in specific cells and granulation tissues. Some reports have it that Substance-P helps reconstruct the cornea when it is damaged. This result was obtained by using Substance-P in a state of being dissolved in a solution. When these solutions are applied, however, they do not remain at an injured site for a long period of time.

A variety of cell formulations are currently used in tissue reconstruction. For example, skin cells, cartilage cells or cardiovascular cells are cultured on a sheet-type scaffold which is applied to an injured site. However, this is problematic because the cells are removed as a sheet-type scaffold from the culture dish during which the cells may be damaged by an enzyme, and the cells may lack to some extent the ability to divide. With the advent of this problem, cell suspensions have attracted keen attention because they are easy to apply and can be easily grafted to even sites where transplantation would be difficult. However, applying cell suspensions requires a bioadhesive such as fibrin because they do not remain there for a desired time but flow down. Therefore, there is a need for a method that allows cells to be reliably applied to injured tissues without interrupting engraftment thereto.

Non-ionic surfactants are not ionized when dissolved in water, ensure wettability, and do not irritate the skin. Thanks to these properties, non-ionic surfactants are used as a cosmetic ingredient, for example, as a dissolving agent for a lotion, as an emulsifier in a cream, and as a cleaning agent in a cleansing cream. In spite of their low cytotoxicity and excellent properties, non-ionic surfactants have nevertheless not been used as vehicles for cell therapy on account of their being acknowledged as inhibiting the engraftment of cells.

The solubility, wettability, emulsifying capacity, and solubilizing capacity of non-ionic surfactants varies depending on the content of lipophilic and hydrophilic groups. Based on this property, non-ionic surfactants may be properly selected depending on the type and concentration and may used in combination with a biomaterial to form compositions suitable to the kind of the drugs or cells to be used and the position of injured tissue.

Korean Patent Laid-Open Publication No. 10-2006-0037176 discloses a wound healing composition containing mesenchymal stem cells and/or Substance-P. This composition, which is nothing but a mixture of one or two ingredients, is apt to migrate from wound sites after application thereto and thus cannot bring about the desired therapeutic effects. Further, the composition is difficult to use. Hence, a method by which the ingredients can be properly delivered to the site of an injury of interest is needed.

Intensive and thorough research into effective cell delivery, conducted by the present invention, resulted in the finding that a hydrogel-type composition containing a non-ionic surfactant, which is used in a broad spectrum of industries, but not in cell therapy, was suitable for use in cell delivery.

The present inventors found that wounds of injured mice healed faster when they had been treated with a hydrogel containing IGF or Substance-P than simply with IGF or Substance-P, when they were treated with a hydrogel containing mesenchymal stem cells rather than simply with mesenchymal stem cells, and when they were treated with a hydrogel containing skin cells rather than simply with skin cells. Accordingly, the present inventors determined the use of hydrogel as a vehicle for cell delivery and completed the present invention.

### SUMMARY OF THE INVENTION

### DISCLOSURE

### Technical Problem

It is therefore an object of the present invention to provide a hydrogel-type cell delivery vehicle composition comprising a non-ionic surfactant.

It is another object of the present invention to provide a hydrogel-type composition for wound healing, comprising a growth factor, Substance-P or cells in addition to the non-ionic surfactant.

It is a further object of the present invention to provide a method for the preparation of the composition.

### Technical Solution

In accordance with an aspect thereof, the present invention provides a hydrogel-type cell delivery vehicle composition comprising a non-ionic surfactant dispersed in an aqueous medium.

Hydrogel is a three-dimensional network of hydrophilic polymer chains that are crosslinked to one another via covalent or non-covalent bonds. Hydrogels can absorb a large amount of water and swell in an aqueous solution or when under an aqueous condition due to their hydrophilic constituents, but do not dissolve due to their crosslink structure. In accordance with the present invention, hydrogel is prepared by dispersing a non-ionic surfactant, a kind of hydrophilic polymer, in an aqueous medium.

As used herein, the term “cell delivery” refers to the delivery of the cells of the composition to a target site of the body, such as the skin, to heal wounds. In this context, the composition serves as a vehicle or carrier for the cells.

So long as it allows hydrophilic non-ionic surfactants to be dispersed therein, any aqueous medium may be employed in the composition of the present invention. Preferably, the aqueous medium is selected from the group consisting of physiological saline, phosphate buffered saline (PBS), and a cell culture medium.

Although it is not electrically charged, the non-ionic surfactant used in the composition of the present invention shows hydrophilicity and forms hydrogen bonds between its hydroxy groups or ethylene oxide groups and water. Examples of the non-ionic surfactant useful in the present invention include polyethylene glycol derivatives, such as ethylene oxide adducts of alkylphenol or higher alcohol, and polyol derivatives prepared by esterifying polyhydroxy compounds such as glycerine, pentaerytritol, sorbitol and saccharose. Preferably, the non-ionic surfactant is selected from among polyethylene glycol condensates such as a fatty acid/polyethyleneglycol condensate (Niosol, Myrj), a fatty acid amide/polyethyleneglycol condensate, a fatty acid alcohol/polyethyleneglycol condensate (Leonil, Peregal C), an aliphatic amind/polyethyleneglycol condensate, an aliphatic mercaptan/polyethyleneglycol condensate (Nyon 218), an alkylphenol/polyethyleneglycol condensate (Igepal), a polypropyleneglycol/polyethyleneglycol condensate (Pluronics) and a combination thereof. Most preferably, the non-ionic surfactant is poloxamer (Pluronic), a polypropyleneglycol/polyethyleneglycol condensate.

The non-ionic surfactant used in the present invention has a hydrocarbon chain ranging in molecular weight from 5,000 to 20,000, with an EO content of 50-80 wt. When the hydrocarbon chain is too short, a satisfactory network structure is not formed. On the other hand, too long of a hydrocarbon chain does not allow the surfactant to disperse in an aqueous medium. The non-ionic surfactant does not form a gel when the EO content is too high, and decreases in hydrophilicity when the EO content is too low.

In the present invention, the composition is prepared by dispersing a non-ionic surfactant in an amount of from 15 to 50 wt % based on the volume of the aqueous medium. When the weight ratio (concentration) of the non-ionic surfactant is too low, it is difficult to form hydrogel. On the other hand, the non-ionic surfactant does not dissolve in an aqueous medium when the weight ratio is too high.

In an embodiment of the present invention, the hydrogel-type composition may further contain a growth factor effective for wound healing, selected from the group consisting of IGF, bFGF, EGF and GMCSF, or Substance-P. The growth factor or Substance-P functions to promote the migration of epithelium cells and the proliferation of fibroblasts.

In another embodiment of the present invention, the hydrogel-type composition may further contain an extracellular matrix (ECM) selected from the group consisting of collagen, hyaluronic acid, glycosaminoglycanes, fibronectin and a mixture thereof. The extracellular matrix functions to increase the adherence of cells which promotes wound healing.

In a further embodiment of the present invention, the hydrogel-type composition may further contain a wound healing-effective biomaterial selected from the group consisting of carboxymethyl cellulose, alginate, chitosan, poly(e-caprolactone), poly(lactic acid), poly(glycolic acid), hydroxyapatite, tricalcium phosphate and a combination thereof. The biomaterial functions to improve hydrogel in property and biocompatibility.

In still another embodiment of the present invention, the hydrogel-type composition may further contain cells. The cells used in the composition of the present invention are delivered to a body side of interest to heal wounds. Examples of the cells useful in the present invention include keratinocytes, fibroblasts, pigment cells, mesenchymal stem cells, mesodermal cells, hematopoietic stem cells, myelocytes, nerve cells, epithermal cells and a combination thereof.

In the composition of the present invention, cell delivery is conducted for wound healing purposes. The term “wound healing”, as used herein, means the treatment or alleviation of the wounds resulting from skin cells having been injured. Once delivered by the composition, the cells substitute for or supplement injured cells at the target site to heal the wound.

Being formulated into a hydrogel, the composition of the present invention can be applied directly to a wound site or administered by injection. The composition may be administered in combination with a pharmaceutically acceptable carrier typically used in cell therapy. The carrier may be physiological saline.

The composition of the present invention is administered in a therapeutically effective amount for wound healing. The term “therapeutically effective amount”, as used herein, is intended to refer to a sufficient amount of the composition to treat a disorder, at a reasonable benefit/risk ratio applicable to any medical treatment. The effective amount may vary depending on various factors including the severity of the disorder being treated, the patient's age and sex, the time of administration, the route of administration, the rate of excretion, the period of time of treatment, the co-administration of drugs, etc. In consideration of these factors, it is important to determine a minimum amount that can bring about the maximum therapeutic effects without producing side effects. This may be readily determined by those skilled in the art. For example, the composition of the present invention may be administered at a single dose of from 1 mg to 1,000 mg for adults. Turning to the basis of cells, MSC may be administered at a single dose of from 3×10⁴ to 3×10⁷ cells/kg.

As will be illustrated in the following Examples, the composition of the present invention was proven to have the capacity of effectively delivering a growth factor, Substance-P and/or cells to wounds because it exerted wetting effects on wounds that prevented the contraction of the wounds (FIGS. 2 to 8), and because it protected cells (FIG. 9). In addition, the composition of the present invention is easy and convenient to use. Further, it is readily conceived that when the non-ionic surfactant of the composition is mixed with a biomaterial such as collagen, a synergistic effect can be obtained. Most preferably, the hydrogel-type composition of the present invention comprises a non-ionic surfactant, a biomaterial, and physiological saline or a cell culture medium at a proper ratio.

### BRIEF DESCRIPTION OF THE DRAWINGS

FIG. 1 shows the change in the viscosity of Pluronic F127 at concentrations of 20%, 25% and 30% with temperature (15-30° C.)

FIG. 2 shows wounds observed with the naked eye on Day 7 after the application of the control (a) and the hydrogel comprising Substance-P (b).

FIG. 3 shows the wounds observed with the naked eye on Day 14 after application of the control (a) and the hydrogel comprising mesenchymal stem cells (b).

FIG. 4 shows histological observations of the wounds on Day 14 after application of the control (a) and the hydrogel comprising mesenchymal stem cells (b).

FIG. 5 shows the wounds observed with the naked eye on Day 7 after the control (a) and the hydrogel comprising skin cells (b) were applied.

FIG. 6 shows histological observations of the wounds on Day 7 after the application of the control (a) and the hydrogel comprising skin cells (b).

FIG. 7 shows the wounds observed with the naked eye on Day 7 after application of the control (a) and the hydrogel comprising IGF (b).

FIG. 8 shows histological observations of the wounds on Day 7 after application of the control (a) and the hydrogel comprising IGF (b).

FIG. 9 is a graph showing the stabilization of skin cells by hydrogel.

### DESCRIPTION OF THE PREFERRED EMBODIMENTS

A better understanding of the present invention may be obtained through the following examples which are set forth to illustrate, but are not to be construed as limiting the present invention.

### EXAMPLE 1

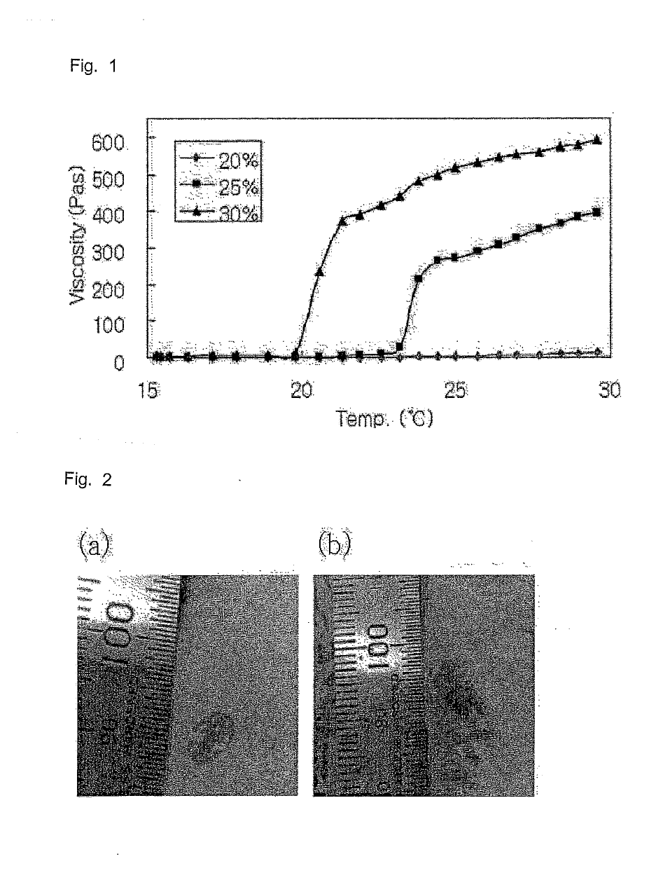

In 50 μL of physiological saline, 12 pmoles of Substance-p and 100 mg of Pluronic F127 (BASF) were mixed to give a hydrogel. Balb/c nude mice (male, 5 weeks old) were injured to produce wounds 8 mm in diameter on their backs. The hydrogel was applied to the wounds while physiological saline was used as a control. Day 7 after application, the wounds were examined with the naked eye. FIG. 2 shows wounds observed with the naked eye on Day 7 after the application of the control (a) and the hydrogel comprising Substance-P (b). As seen, the hydrogel of the present invention exerted a wetting effect on the wound and suppressed the contraction of the wound, thus effectively promoting wound healing, compared to the control.

In 10 mL of physiological saline were dissolved 2 g, 2.5 g and 3 g of Pluronic F127 to prepare 20%, 25% and 30% hydrogels, respectively. These hydrogels were monitored for change in viscosity with temperature (15-30° C.) using a rheometer (CVO, BOHLIN Instruments). FIG. 1 shows the change of the viscosity of Pluronic F127 at concentrations of 20%, 25% and 30% with temperature (15-30° C.). As shown, the property of hydrogel varies depending on the concentration of the non-ionic surfactant. When the hydrogel is injected to the body to regenerate the tissue, a concentration at which the viscosity can be changed with temperature is more advantageous. On the other hand, when it is applied topically or to the skin, the hydrogel can be used irrespective of the change of viscosity with temperature.

### EXAMPLE 2

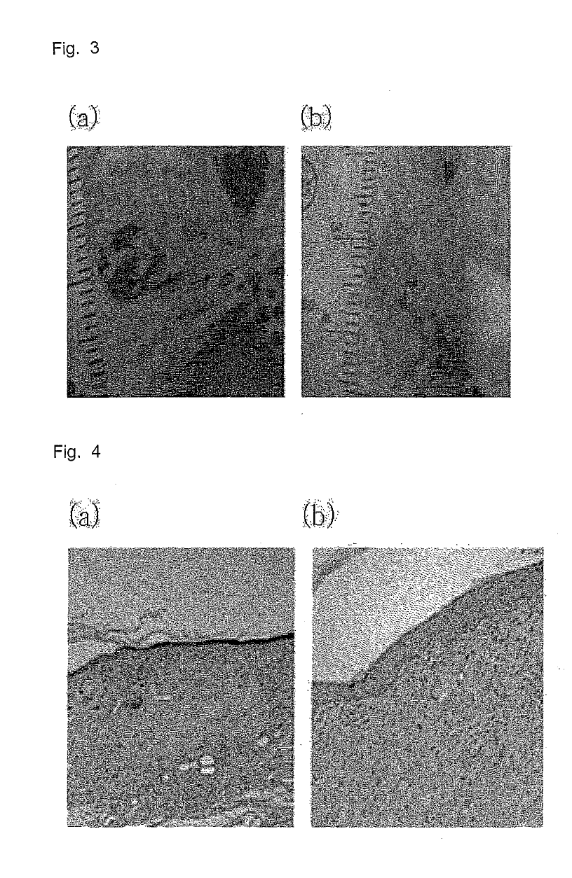

In 50 μL of a mesenchymal stem cell (MSC) growth medium (MSCGM), 1×10⁶ mesenchymal stem cells and 100 mg of Pluronic F127 were mixed to give a hydrogel. To an 8 mm-diameter wound formed on the back of a Balb/c nude mouse (male, 5 weeks old) was applied 50 μL of the hydrogel while physiological saline was used as a control. On Day 6 after application, the same hydrogel was applied again. On Day 14 after the initial application, the wounds on the back of the mice were observed with the naked eye and examined histologically. FIG. 3 shows the wounds observed with the naked eye on Day 14 after the application of the control (a) and the hydrogel comprising mesenchymal stem cells (b). FIG. 4 shows histological observations of the wounds on Day 14 after the control (a) and the hydrogel comprising mesenchymal stem cells (b) were applied. As seen from the observations with the naked eye, the hydrogel of the present invention exerted a wetting effect on the wound and suppressed the contraction of the wound, thus effectively promoting wound healing, compared to the control. In addition, the histological observations demonstrate that the epidermis and the dermis were better established in the experimental group than in the control.

### EXAMPLE 3

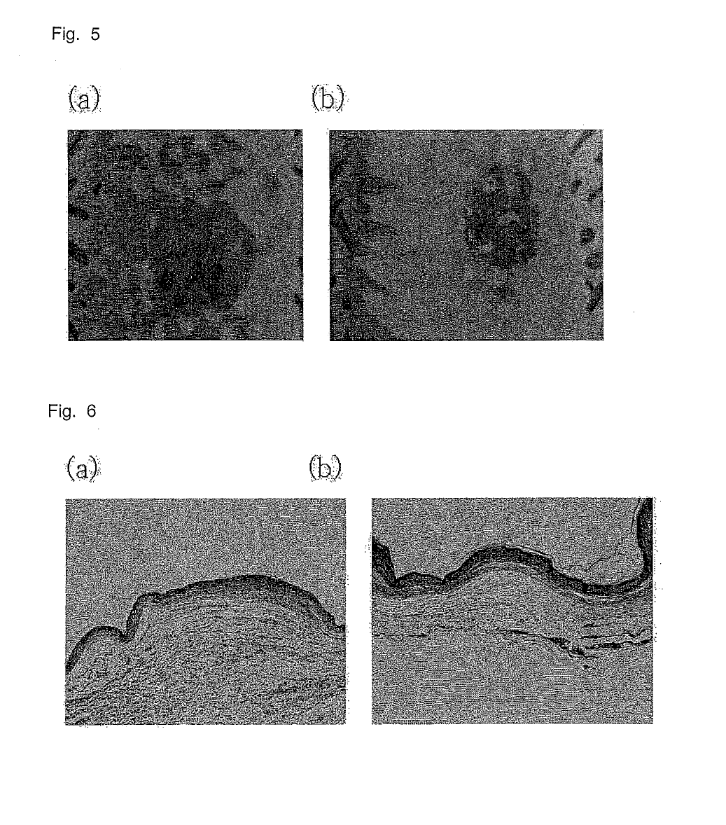

In 50 μL of a skin cell culture medium (DMEM), 5×10⁵ skin cells (fibroblasts, keratinocytes and pigment cells) and 100 mg of Pluronic F127 were mixed to give a hydrogel. To an 8 mm-diameter wound formed on the back of a Balb/c nude mouse (male, 5 weeks old) was applied 50 μL of the hydrogel while physiological saline was used as a control. On Day 7 after application, the wounds on the back of the mice were observed with the naked eye and examined histologically. FIG. 5 shows the wounds observed with the naked eye on Day 7 after the control (a) and the hydrogel comprising skin cells (b) were applied. FIG. 6 shows histological observations of the wounds on Day 7 after the control (a) and the hydrogel comprising skin cells (b) were applied. As seen from the observations with the naked eye, the hydrogel of the present invention exerted a wetting effect on the wound and suppressed the contraction of the wound, thus effectively promoting wound healing, compared to the control. In addition, the histological observations demonstrate that the epidermis and the dermis were better established in the experimental group than in the control.

### EXAMPLE 4

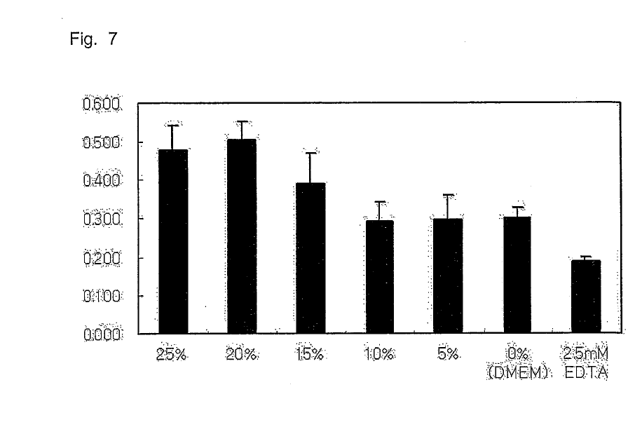

In 50 μL of physiological saline, 25 μg/mL IGF (insulin like growth factor) and 100 mg of Pluronic F127 (BASF) were mixed to give a hydrogel. To an 8 mm-diameter wound formed on the back of a Balb/c nude mouse (male, 5 weeks old) was applied 50 μL of the hydrogel while physiological saline was used as a control. On Day 7 after application, the wounds on the back of the mice were observed with the naked eye and examined histologically. FIG. 7 shows the wounds observed with the naked eye on Day 7 after the control (a) and the hydrogel comprising IGF (b) were applied. FIG. 8 shows histological observations of the wounds on Day 7 after the control (a) and the hydrogel comprising IGF (b) were applied. As seen from the observations with the naked eye, the hydrogel of the present invention exerted a wetting effect on the wound and suppressed the contraction of the wound, thus effectively promoting wound healing, compared to the control. In addition, the histological observations demonstrate that the epidermis and the dermis were better established in the experimental group than in the control.

### EXAMPLE 5

Skin cells (fibroblasts, keratinocytes and pigment cells) were seeded at a density of 2×10⁴ cells/well in 96-well plates and cultured at 37° C. for 16 hrs. After removal of the medium, hydrogel was diluted at various concentrations in a skin cell culture medium and added to each well. As a control, 100 μL of 2.5 mM EDTA was added. The cells were incubated at 4° C. for 16 hrs, followed by the removal of the medium from each well. A mixture of 1:9 MTT solution:cell culture medium was added to each well and incubated at 37° C. for 4 hrs. The cells were washed with PBS and incubated for 20 min in a mixture of 1:1 DMSO:ethanol, followed by measuring absorbance at 540 nm. FIG. 9 is a graph showing the stabilization of skin cells by hydrogel. At 4° C., cell stability was increased in the presence of hydrogel, compared to the control (DMEM), and particularly 1.5-fold increased upon the addition of 20 or 25% hydrogel, compared to the control.

### INDUSTRIAL APPLICABILITY

As described hitherto, the hydrogel-type composition of the present invention can effectively deliver a growth factor, Substance-P and/or cells to wounds and has the function of exerting wetting effects on wounds to prevent the contraction of the wounds (FIGS. 2 to 8), and protecting cells (FIG. 9). In addition, the composition of the present invention is easy and convenient to use. Therefore, the composition of the present invention can deliver its cells to injured sites, promoting wound healing when it is applied or injected to the injured sites.

1-11. (canceled)

12. A hydrogel-type cell delivery vehicle composition, comprising an aqueous medium in which a non-ionic surfactant is dispersed in an amount of 15˜50 wt % based on a total weight of the composition.

13. The hydrogel-type cell delivery vehicle composition as set forth in claim 12, wherein the non-ionic surfactant is selected from the group consisting of a fatty acid/polyethyleneglycol condensate, a fatty acid amide/polyethyleneglycol condensate, an aliphatic alcohol/polyethyleneglycol condensate, an aliphatic amine/polyethyleneglycol condensate, an aliphatic mercaptan/polyethyleneglycol condensate, an alkylphenol/polyethyleneglycol condensate, a polypropyleneglycol/polyethyleneglycol condensate and a combination thereof.

14. The hydrogel-type cell delivery vehicle composition as set forth in claim 13, wherein the non-ionic surfactant is Poloxamer, a polypropyleneglycol/polyethyleneglycol condensate.

15. The hydrogel-type cell delivery vehicle composition as set forth in claim 12, further comprising a wound healing-effective growth factor selected from among IGF, bFGF, EGF and GMCSF, or Substance-P.

16. The hydrogel-type cell delivery vehicle composition as set forth in claim 12, further comprising a wound healing-effective extracellular matrix (ECM) selected from the group consisting of collagen, hyaluronic acid, glycosaminoglycanes, fibronectin and a combination thereof.

17. The hydrogel-type cell delivery vehicle composition as set forth in claim 12, further comprising cells.

18. The hydrogel-type cell delivery vehicle composition as set forth in claim 12, wherein the cells are selected from the group consisting of keratinocytes, fibroblasts, pigment cells, mesenchymal stem cells, mesodermal stem cells, hemopoietic stem cells, myelocytes, nerve cells, epithelial cells and a combination thereof.

19. The hydrogel-type cell delivery vehicle composition as set forth in claim 12, being used in wound healing.
